(19) United States
(12) Patent Application Publication
(10) Pub. No.: US 2004/0153130 A1
(43) Pub. Date: Aug. 5, 2004

(54) Methods for treating muscular dystrophy

(76) Inventors: Amir Oron, Tel-Aviv (IL); Jackson Streeter, Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/448,262

(22) Filed: May 29, 2003

Related U.S. Application Data
(63) Continuation-in-part of application No. 10/287,432, filed on Nov. 1, 2002.
(60) Provisional application No. 60/384,050, filed on May 29, 2002.

(51) Int. Cl. A61N 5/06

(52) U.S. Cl. 607/88; 607/89

(57) ABSTRACT

Therapeutic methods for treating or inhibiting a neuromuscular disease or condition, including muscular dystrophy, in a subject in need thereof are described, the methods including applying to muscle tissue of the subject a muscular dystrophy effective amount of electromagnetic energy having a wavelength in the visible to near-infrared wavelength. In a preferred embodiment, the muscular dystrophy effective amount of energy comprises predetermined power density (mW/cm²) of the electromagnetic energy of at least 1 mW/cm², which is provided from a laser or other light energy source.

### Related Application Information

This application claims priority under 35 U.S.C. § 119(e) to U.S. Provisional Application Serial No. 60/384,050, filed May 29, 2002 and is a continuation-in-part of U.S. patent application Ser. No. 10/287,432, filed Nov. 1, 2002, the disclosures of which are hereby incorporated by reference in their entireties.

### BACKGROUND OF THE INVENTION

1. Field of the Invention

The present invention relates in general to therapeutic methods for the treatment of muscular dystrophy, and more particularly to methods for treating muscular dystrophy by applying electromagnetic energy.

2. Description of the Related Art

Muscular dystrophy (MD) encompasses a group of genetically determined muscular disorders that are characterized by progressive wasting and weakness of the skeletal muscle, and often also of the cardiac and smooth muscles or other tissues. See, e.g., K. Arhata, NEUROPATHOLOGY 20:34-41 (2000); C. Angelini and D. M. Bonifati, Neurol. Sci. 21: 919-24 (2002).

Duchenne muscular dystrophy (DMD) is a particularly devastating form of muscular dystrophy. Children affected with DMD are typically confined to a wheelchair by the age of 12 years, are bedridden by their twenties, and die before the age of thirty. The pathogenetic mutation that leads to DMD has been identified, and results in loss of the subsarcolemmal protein dystrophin. In the MD variant known as Becker muscular dystrophy (BMD), different mutations in the dystrophin gene result in some dystrophin production, but in insufficient quantity or quality, although having some dystrophin protects the muscles of those with BMD from degenerating as badly or as quickly as individuals with DMD. A number of other new genes have been discovered that cause different genetic forms of MD, and it appears that multiple but overlapping disease mechanisms might be involved, all leading to the final common pathway of cell death in these disorders.

However, the cellular and molecular mechanisms underlying MD, even for those forms of the disorder for which a genetic basis has been identified, remain unclear. For example, the precise role of dystrophin in MD is unclear. Initial research suggests that a dystrophin-glycoprotein complex has both mechanical and signaling roles, but additional study will be required to determine the potential role of mediators that may be involved with the dystrophic process. Other recent work suggests that novel alterations of the cell cytoskeleton may be involved in DMD.

Thus, current research on muscular dystrophy and other neuromuscular diseases includes a variety of strategies and approaches that have yet to lead to fully satisfactory treatment options. Curative therapy is not yet available. Currently, treatment for the muscular dystrophies typically involves administration of steroids, which are generally considered to be the most effective treatment option available for DMD. However, steroids generally have a brief and transient effect and are associated with numerous complications. In addition, despite the effectiveness of steroids in treating DMD, the specific immune response and nature of the inflammatory changes that accompany degeneration in DMD, as well as in other muscular dystrophies and other neuromuscular disorders, are not yet well understood. The role of corticosteroids in controlling inflammation in DMD, and the mechanism of action of corticosteroids on muscle cell stability and function and on stem cells have yet to be elucidated. U.S. Pat. No. 5,621,091 describes a method of therapy for MD that involves the insertion of cDNA corresponding to the MD gene, or a fragment thereof, into a vector, and reintroducing these genetically altered cells back into the subject. However, gene-replacement therapy is still in the early stages of development.

High energy laser radiation is now well accepted as a surgical tool for cutting, cauterizing, and ablating biological tissue. High energy lasers are now routinely used for vaporizing superficial skin lesions and, to make deep cuts. For a laser to be suitable for use as a surgical laser, it must provide laser energy at a power sufficient to heat tissue to temperatures over 50° C. Power outputs for surgical lasers vary from 1-5 W for vaporizing superficial tissue, to about 100 W for deep cutting.

In contrast, low level laser therapy involves therapeutic administration of laser energy to a patient at vastly lower power outputs than those used in high energy laser applications, resulting in desirable biostimulatory effects while leaving tissue undamaged. In rat models of myocardial infarction and sichemia-reperfusion injury, low energy laser irradiation reduces infarct size and left ventricular dilation, and enhances angiogenesis in the myocardium. (Yaakobi et al., J. Appl. Physiol. 90,2411-19 (2001)). Low level laser therapy has been described for treating pain, including headache and muscle pain, and inflammation following cold or physical trauma or injury. See, e.g., Belkin et al., Lasers Light Ophthalmol. 2:63-71 (1988).

Against this background, a high level of interest remains in finding new and improved therapeutic methods for the treatment of muscular dystrophy.

### SUMMARY OF THE INVENTION

In accordance with one embodiment, there is provided a method for treating or inhibiting neuromuscular disease or condition, including muscular dystrophy, in a subject in need of such treatment or inhibition. The method comprises applying to an area of skin overlying target muscle tissue of the subject a muscular dystrophy effective amount of electromagnetic energy having a wavelength in the visible to near-infrared wavelength range, wherein the muscular dystrophy effective amount of electromagnetic energy has a power density sufficient to achieve a biostimulatory effect on the target muscle tissue. In a preferred embodiment, applying the muscular dystrophy effective amount of electromagnetic energy comprises applying a predetermined power density of electromagnetic energy to the skin, wherein the predetermined power density is calculated taking into account attenuation of the energy applied to the skin by tissue lying between the skin and the target muscle tissue.

In accordance with another embodiment, there is provided a method for treating or inhibiting a muscular dystrophy (MD) condition in a subject in need of such treatment or inhibition. The method comprises inserting a cDNA, or a fragment thereof, corresponding to the MD gene into a vector to form genetically altered cells, applying an amount of electromagnetic energy to the genetically altered cells sufficient to achieve a biostimulatory effect, said electromagnetic energy having a wavelength in the visible to near-infrared wavelength range, and introducing the genetically altered cells into the subject. The introduction of the cells to the subject may occur before and/or after the application of energy to the cells.

### BRIEF DESCRIPTION OF THE DRAWINGS

FIG. 1 is a perspective view of a first embodiment of a light therapy device; and

FIG. 2 is a block diagram of a control circuit for a light therapy device, such as is illustrated in FIG. 1.

### DETAILED DESCRIPTION OF THE PREFERRED EMBODIMENT

The methods described herein may be practiced using, for example, a low level laser therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference in their entireties together with the references contained therein. Such apparatus, and other suitable apparatus, preferably include light energy sources, such as laser light sources, capable of emitting light energy having a wavelength in the visible to near-infrared wavelength range, preferably from about 630 nm to about 904 nm, including below about 820 nm. A handheld probe may be used for delivering the laser or light energy. The probe includes a laser source of light energy. The probe may include, for example, a single laser diode that provides about 100 mW to about 500 mW of total power output, or multiple laser diodes that together are capable of providing at least about 100 mW to about 500 mW of total power output. In preferred apparatus, the actual power output is variable using control unit electronically coupled to the probe, so that the power of the laser energy emitted can be adjusted in accordance with power density calculations as described infra.

The methods described herein preferably use electromagnetic energy having a wavelength in the visible to near-infrared wavelength range below about 820 nm. In one embodiment, the wavelength is in the range of about 700 nm to about 800 nm, a range of wavelengths that appears to be especially suitable for obtaining desired effects on cells. In another embodiment, the wavelength is in the range of about 725 nm to about 785 nm. In one exemplary embodiment, the wavelength is about 730 nm, and in another exemplary embodiment, the wavelength is about 780 nm. Examples of suitable light sources for producing the electromagnetic energy include laser sources such as the semiconductor, continuously emitting GaAIAs laser (emitting at about 780 nm), and the crystalline pulsed lasers Alexandrite (emitting at 755 nm) and Ti:sapphire (emitting at 700-900 nm). Alternatively, the electromagnetic energy source is another type of diode, for example light-emitting diode (LED), or other light energy source, provided that the electromagnetic energy source has a wavelength in the visible to near-infrared wavelength range, below about 820 nm, preferably in the range of about 700 nm to about 800 nm, including from about 725 nm to about 785 nm, and about 730 nm or about 780 nm. The level of coherence of a light energy source is not critical. A light energy source used as the electromagnetic energy source need not provide light having the same level of coherence as the light provided by a laser energy source and/or it may be substantially non-coherent.

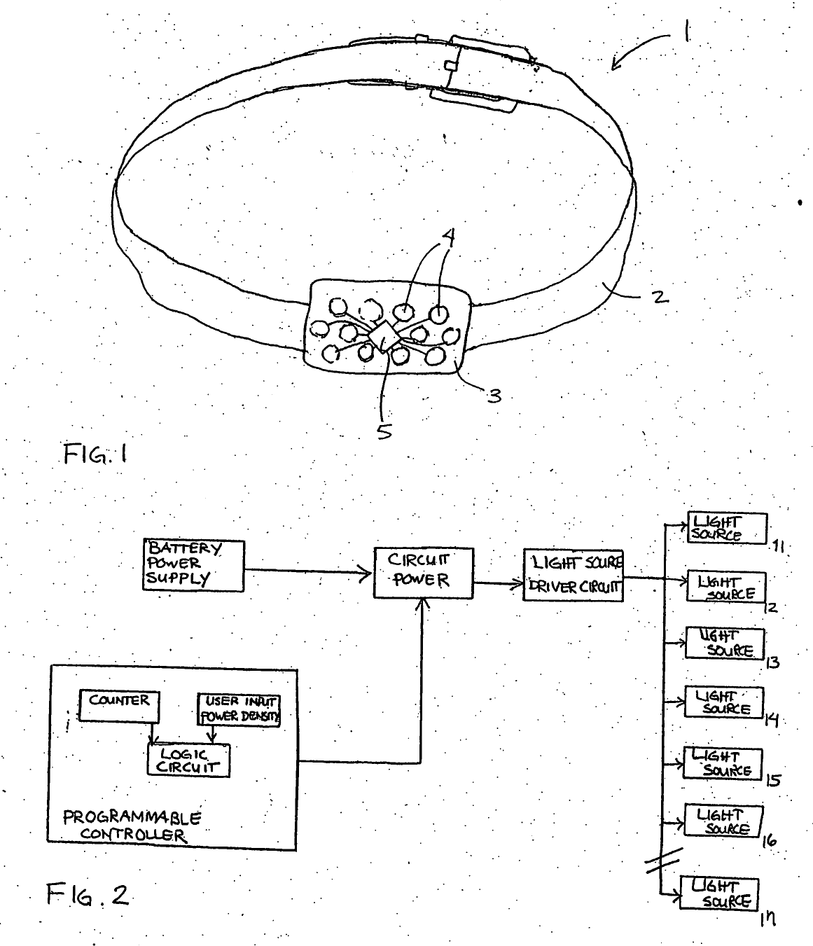

Another suitable light therapy apparatus is that illustrated in FIG. 1. The device of FIG. 1 is especially useful for transdermal applications of light energy. The illustrated device 1 includes a flexible strap 2 with a securing means, the strap adapted for securing the device over an area of the subject's body, one or more light energy sources 4 disposed on the strap 2 or on a plate or enlarged portion of the strap 3, capable of emitting light energy having a wavelength in the visible to near-infrared wavelength range, a power supply operatively coupled to the light source or sources, and a programmable controller 5 operatively coupled to the light source or sources and to the power supply. Based on the surprising discovery that control or selection of power density of light energy is an important factor in determining the efficacy of light energy therapy, the programmable controller is configured to select a predetermined surface power density of the light energy sufficient to deliver a predetermined subsurface power density to a body tissue to be treated beneath the skin surface of the area of the subject's body over which the device is secured.

The light energy source or sources are capable of emitting the light energy at a power sufficient to achieve a predetermined power density. The strap is preferably fabricated from an elastomeric material to which is secured any suitable securing means, such as mating Velcro strips, snaps, hooks, buttons, ties, or the like. Alternatively, the strap is a loop of elastomeric material sized appropriately to fit snugly over a particular body part, such as a particular arm or leg joint, or around the chest or hips. Non-elastomeric strap materials may also be used. The precise configuration of the strap is subject only to the limitation that the strap is capable of maintaining the light energy sources generally in a position relative to the particular area of the body or tissue being treated. In an alternative embodiment, a strap is not used and instead the light source or sources are incorporated into or attachable onto a piece of fabric which is draped over the target body portion or fits securely over the target body portion thereby holding the light source or sources in proximity to the patient's body for treatment. The fabric used is preferably a stretchable fabric or mesh comprising materials such as Lycra or nylon, but other fabrics and materials may be used as well, including substantially non-stretchable fabrics and materials. The light source or sources are preferably removably attached to the fabric so that they may be moved and placed in the position needed for treatment.

In the exemplary embodiment illustrated in FIG. 1, a light therapy device includes a flexible strap and securing means such as mating Velcro strips configured to secure the device around the body of the subject. The light source or sources are disposed on the strap, and in one embodiment are enclosed in a housing secured to the strap. Alternatively, the light source or sources are embedded in a layer of flexible plastic or fabric that is secured to the strap. In any case, the light sources are preferably secured to the strap so that when the strap is positioned around a body part of the patient, the light sources are positioned so that light energy emitted by the light sources is directed toward the skin surface over which the device is secured. Various strap configurations and spatial distributions of the light energy sources are contemplated so that the device can be adapted to treat different tissues in different areas of the body.

FIG. 2 is a block diagram of a control circuit according to one embodiment of the light therapy device. The programmable controller is configured to select a predetermined surface power density of the light energy sufficient to deliver a predetermined power density to the target area. The actual total power output if the light energy sources is variable using the programmable controller so that the power of the light energy emitted can be adjusted in accordance with what is calculated as being needed for treatment.

Preferred light energy source or sources used in the preferred methods herein are capable of emitting the light energy at a power sufficient to achieve a predetermined subsurface power density. The subsurface power density is the power density seen at the target tissue, taking into account attenuation of the energy as it travels through skin, bone, other body tissue, and fluids from the surface to the target tissue. It is presently believed that tissue will be most effectively treated using subsurface power densities of light of at least about 0.01 mW/cm² and up to about 100 mW/cm², including about 0.05, 0.1, 0.5, 1, 5, 10, 15, 20, 30, 40, 50, 60, 70, 80, and 90 mW/cm². In one embodiment, power densities of about 20 mW/cm² to about 50 mW/cm² are used. To attain subsurface power densities within these stated ranges, taking into account attenuation of the energy as noted above, surface power densities of from about 1 mW/cm² to about 500 mW/cm² are needed in most circumstances. In some circumstances, depending upon the degree of attenuation of the energy as it travels from the source to the target tissue, surface power densities above and below this range may also be used. To achieve such surface power densities, preferred light energy sources, or light energy sources in combination, are capable of emitting light energy having a total power output of at least about 1 mW to about 500 mW, including about 5, 10, 20, 30, 50, 75, 100, 150, 200, 250, 300, and 400 mW, but may also be up to about 1000 mW. It is believed that the subsurface power densities of at least about 0.01 mW/cm² and up to about 100 mW/cm² in terms of the power density of energy that reaches the subsurface tissue are especially effective at producing the desired biostimulative effects on tissue being treated. Although the aforementioned values are listed as preferred, other power densities, surface and subsurface, and power outputs may also be used in accordance with the methods described herein.

Neuromuscular conditions and diseases that can be treated according to the electromagnetic energy therapy methods include, for example, the muscular dystrophies Duchenne Muscular Dystrophy (DMD), Becker Muscular Dystrophy (BMD), Outlier Muscular Dystrophy (OMD), Emery-Dreifuss Muscular Dystrophy (EDMD), Limb-Girdle Muscular Dystrophy (LGMD), Facioscapulohumeral Muscular Dystrophy (FSH or FSHD; also known as Landouzy-Dejerine), Myotonic Dystrophy (MMD; also known as Steinert's Disease); Oculopharyngeal Muscular Dystrophy (OPMD) Distal Muscular Dystrophy (DD) and Congenital Muscular Dystrophy (CMD), as well as other neuromuscular diseases that involve or can involve voluntary muscle cell death or inflammation, including the myositis disorders polymyositis, dermamyositis and inclusion body myositis, as well as myopathies. In general, the methods can be used to treat the voluntary muscles of a subject having any muscular or neuromuscular disorder involving muscle weakness or wasting, whether the primary cause is genetic, autoimmune or another factor.

Preferred methods are based at least in part on the finding that the power density of the light energy (i.e., light intensity or power per unit area, in W/cm²) delivered to tissue appears to be a very important factor in determining the relative efficacy of therapy. Without being bound by theory, it is believed that light energy delivered within a certain range of power densities provides the required biostimulative effect on the intracellular environment, such that the function of previously nonfunctioning or poorly functioning mitochondria in target cells is, enhanced so as to return to a more normal state and the functioning of normally functioning mitochondria in neurons is enhanced to achieve better than normal functioning, such functioning supporting the basic cellular functions and activity required for growth, repair, regeneration, differentiation and reproduction. The biostimulatory effects on cells can also result in prevention or inhibition of apoptotic or necrotic processes that occur secondarily to a primary disease, condition or insult to the tissue. In particular, for the treatment of a subject suffering from muscular dystrophy, muscle cells treated with electromagnetic energy according to the present methods will resist necrosis and regain conductive and contractile function. More particularly, the power density electromagnetic energy applied to an area of skin overlying a muscle to be treated, independent of the power of the electromagnetic energy source used and the dosage of the energy used, appears to enhance basic biological functions that support cell growth, differentiation and reproduction.

As used herein, the terms “biostimulative” and “biostimulatory” as used herein refer to a characteristic of an amount of electromagnetic energy delivered to cells in vivo, or in vitro, wherein the electromagnetic energy enhances basic cell biological functions such as respiration, protein synthesis and transport, intracellular and intracellular signaling, and cellular metabolism, that underlie cell activity involved in cell growth, repair, regeneration, differentiation and reproduction. The biostimulatory effect can be seen as improvement of a patient's condition due to diminution of the symptoms of a neuromuscular disease or condition from which the patient suffers.

A muscular dystrophy (or neuromuscular disease) effective amount of electromagnetic energy as used herein is a surface power density (mW/cm²) of electromagnetic energy applied to the cells or tissue being treated. The surface power density is sufficient to achieve a desired power density of energy to the target cells or muscle tissue that produces biostimulatory effects. In the case of treatment that occurs through the skin or other tissue of a patient, the surface power density may be calculated from the desired power density to be delivered to the cells or tissue, taking into account factors that attenuate the energy as it travels from the skin surface to the cells or tissue being treated. Such factors include the amount of tissue such as fat or other organ tissue intervening between the area of skin at which the energy is applied and target muscle, and degree of skin pigmentation wherein darker, more heavily pigmented skin absorbs more energy and therefore would require a higher surface power density. For example, to obtain a desired power density of 50 mW/cm² at a target at a depth of 3 cm below the surface may require a surface power density of 500 mW/cm².

In particular, according to the present methods for treating MD, the electromagnetic energy is applied to a region or are of skin adjacent to a muscle to be treated such as a skeletal muscle such as a hand, arm or leg muscle. The term “adjacent” in the foregoing context means that the area of skin overlies the muscle to be treated, whether or not intervening tissue such as fat, other muscle or other organs lies between the area of skin and the muscle to be treated, provided only that the area of skin is sufficiently located that a beam of electromagnetic energy applied to the area of skin is directed toward the target muscle. Other muscles that are sometimes affected in MD, including cardiac muscle and smooth muscle, can also be treated.

Thus, one embodiment of method for treating or inhibiting MD or other neuromuscular disease or condition in a subject involves delivering biostimulative energy having a wavelength in the visible to near-infrared wavelength range to target cells or tissue in the subject. The power density to be delivered to the tissue is selected to be at least about 0.01 mW/cm². In one embodiment, the power density is selected from the range of about 1 mW/cm² to about 100 mW/cm². In a preferred embodiment, delivering the biostimulative energy includes delivering an MD effective amount of light energy, which involves selecting a surface power density of the light energy sufficient to deliver the predetermined MD effective subsurface power density of light energy to the target cells or tissue. The surface power density may be selected by performing a calculation which takes into account attenuating factors such as those described above. The power and other parameters are then adjusted according to the results of the calculation.

In preferred embodiments, the power density at the target cells or tissue (which, in most in vivo embodiments is the subsurface power density) to be delivered to the tissue is selected to be at least about 0.01 mW/cm², including from about 1 mW/cm² to about 100 mW/cm². The precise power density selected depends on a number of factors, including the specific wavelength of light selected, the type of disease, the clinical condition of the subject, and the like. Similarly, it should be understood that the power density of light energy to be delivered to the affected tissue may be adjusted to be combined with any other therapeutic agent or agents, especially pharmaceutical agents to achieve the desired biological effect.

The wavelength of the light energy is preferably selected from the range of about 630 nm to about 904 nm. In one embodiment, using light apparatus including GaAIAs laser diodes, the light energy has a wavelength of about 830 nm. In some embodiments, more than one wavelength may be used, such wavelengths being delivered substantially simultaneously or in being delivered in series, such as by using a source that scans through the wavelengths.

In preferred embodiments, the light source used in light therapy is a coherent source (i.e. a laser), and/or the light is substantially monochromatic (i.e. one wavelength or a very narrow band of wavelengths).

In preferred embodiments, the treatment proceeds continuously for a period of about 30 seconds to about 2 hours, more preferably for a period of about 1 to 20 minutes. A treatment period may occur once daily, several times daily, on alternate days, or on another basis as deemed appropriate by the therapist or physician. In one embodiment, treatment occurs at least once per day initially for at least 2-3 days, and continues for weeks, months, or indefinitely for as long as a trained therapist or physician determines that muscle function is improving or at least that loss of function is arrested. The irradiation therapy can also be repeated on a daily, several-times daily, or alternate day basis or at other intervals The length of treatment time and frequency of treatment periods may be determined by the trained therapist or physician to result in optimal therapeutic effects for the patient, considering various clinical factors such as the severity and stage of the MD or other neuromuscular disease, age of the subject, presence of other diseases or conditions, effectiveness of drug therapy, and the like.

During the treatment, the light energy may be continuously provided, or it may be pulsed. If the light is pulsed, the pulses are preferably at least about 10 ns long and occur at a frequency of up to about 100 Hz. Continuous wave light may also be used.

In one embodiment, the area of skin adjacent to an affected muscle is irradiated with electromagnetic energy having a wavelength in the visible to near-infrared wavelength range. In one embodiment, scanning electromagnetic energy is used, and the energy source has a power output of about 50 mW to about 500 mW. The energy is applied to the skin at an approximate power density of at least about 0.01 mW/cm², including about 1 mW/cm² to about 100 mW/cm² and about 2 mW/cm² to about 20 mW/cm². In an exemplary embodiment, the electromagnetic energy is applied to an area of skin adjacent a muscle to be treated using a scanning energy beam at a speed of about 2 cm per sec for a duration of 20 min. every alternate day for a period of 2 months.

The energy therapy methods as described herein can also be advantageously used in combination with gene therapy to regenerate skeletal muscle tissue or other tissue affected by a dystrophin gene mutation. For example, as described in U.S. Pat. No. 5,621,091 (the disclosure of which is herein incorporated by reference), a method of therapy for MD involves the insertion of cDNA, or a fragment thereof, corresponding to the MD gene into a vector, and introducing (including reintroducing in the case of cells which originated with the subject) these genetically altered cells into the subject. The cells are injected into the bloodstream or muscle tissue to produce dystrophin in an amount effective to control the degeneration of muscle fibers and to control the proliferation of connective tissue within the muscle fibers. In one embodiment of the present methods, the gene therapy approach to treating MD is combined with light energy therapy, wherein the light energy therapy is applied directly to the genetically altered cells in vitro before introduction of the cells into the subject and/or applied directly to the cells in vivo after introduction of the genetically altered cells into the subject. Light energy therapy may also be applied indirectly to the genetically altered cells and other cells in vivo by applying the light energy to skin overlying the location of the target cells in vivo.

Application of light in vitro may be done by irradiating cells in culture using a hand-held light delivery apparatus according to the principles discussed herein, wherein the subsurface power density is roughly equivalent to the surface power density. Specialized apparatus for in vitro treatment of cells is described in U.S. Provisional Application Serial No. 60/423,643, entitled Enhancement of In Vitro Culture Using Electromagnetic Energy Treatment, filed Nov. 1, 2002, the disclosure of which is hereby incorporated by reference in its entirety.

Therefore, in another aspect, the present methods include administering cDNA, or a fragment thereof, corresponding to the MD gene into a vector, reintroducing genetically altered cells back into the subject, and applying a biostimulatory amount of electromagnetic energy having a wavelength in the visible to near-infrared wavelength range to the genetically altered cells. The combined genetic and light energy therapy may also be used in combination with local application by injection, surgical implantation, instillation or any other means, of other therapeutic agents such as steroids or other agents that provide therapeutic effects for MD patients.

### EXAMPLE 1

A 14-year-old male with a diagnosis of Duchenne muscular dystrophy and wheelchair bound (already in a wheelchair) was treated noninvasively with low energy laser irradiation. Electromyography analysis of nerve conductivity and muscle strength was performed on leg and hand muscles prior to treatment. Muscle strength was also analyzed by subjective physical test performed by a neurologist. Hand or leg muscles were irradiated with scanning He-Ne laser applied to overlying skin at a power output of 50 mW, and at an approximate power density on the muscle of 4 mW/cm². The scanning beam was applied at a speed of 2 cm per sec for a duration of 20 min. every alternate day for a period of 2 months. One leg or hand was irradiated while the correlated hand or leg served as control. EMG was preformed 2 weeks after final irradiation. EMG indicated a 76% increase in hand and leg nerve conductance and 52% increase in muscle contraction. Low energy irradiation thus improved the performance of muscle activity in an acute case of Duchenne muscular dystrophy.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for treating or inhibiting neuromuscular disease or condition, including muscular dystrophy, in a subject in need of such treatment or inhibition, said method comprising applying to an area of skin overlying a target muscle tissue of the subject a muscular dystrophy effective amount of electromagnetic energy having a wavelength in the visible to near-infrared wavelength range, wherein the muscular dystrophy effective amount of electromagnetic energy has a power density sufficient to achieve a biostimulatory effect on the target muscle tissue.

2. The method according to claim 1 wherein applying the muscular dystrophy effective amount of electromagnetic energy comprises applying a predetermined power density of electromagnetic energy to the skin, wherein the predetermined power density is calculated taking into account attenuation of the energy applied to the skin by tissue lying between the skin and the target muscle tissue.

3. The method according to claim 1 wherein the muscle is a skeletal muscle of the subject.

4. The method according to claim 2 wherein the predetermined power density is a power density selected to achieve a subsurface power density of at least about 0.01 mW/cm².

5. The method according to claim 2 wherein the predetermined power density is a power density selected from the range of about 1 mW/cm² to about 100 mW/cm².

6. The method according to claim 5 wherein the predetermined power density is selected from the range of about 20 mW/cm² to about 50 mW/cm².

7. The method according to claim 1 wherein the electromagnetic energy has a wavelength of about 630 nm to about 904 run.

8. The method according to claim 7 wherein the electromagnetic energy has a wavelength of about 830 nm.

9. The method according to claim 1 wherein applying the muscular dystrophy effective amount of electromagnetic energy further comprises providing a laser energy source for generating the electromagnetic energy.

10. The method according to claim 1 wherein applying the muscular dystrophy effective amount of electromagnetic energy further comprises providing a non-coherent light source for generating the electromagnetic energy.

11. The method according to claim 1 further comprising a continuous light source for generating the electromagnetic energy.

12. The method according to claim 1 further comprising a pulsed light source for generating the electromagnetic energy.

13. A method for treating or inhibiting a muscular dystrophy (MD) condition in a subject in need of such treatment or inhibition, said method comprising:
inserting a cDNA, or a fragment thereof, corresponding to the MD gene into a vector to form genetically altered cells;
applying an amount of electromagnetic energy to the genetically altered cells sufficient to achieve a biostimulatory effect, said electromagnetic energy having a wavelength in the visible to near-infrared wavelength range; and
introducing the genetically altered cells into the subject.

14. The method according to claim 13 wherein applying the biostimulatory effective amount of electromagnetic energy comprises delivering a predetermined power density of electromagnetic energy to the genetically altered cells.

15. The method according to claim 14 wherein the predetermined power density is a power density of at least about 0.01 mW/cm².

16. The method according to claim 14 wherein the predetermined power density is a power density selected from the range of about 1 mW/cm² to about 100 mW/cm².

17. The method according to claim 14 wherein the electromagnetic energy has a wavelength of about 630 nm to about 904 nm.

18. The method according to claim 14, wherein the electromagnetic energy has a wavelength of about 830 nm.

19. The method according to claim 13, wherein the applying of electromagnetic energy is performed following introduction of the cells into the subject.

20. The method according to claim 13, wherein the applying of electromagnetic energy is performed prior to introduction of the cells into the subject.
